(19) United States
(12) Patent Application Publication
(10) Pub. No.: US 2026/0199192 A1
(43) Pub. Date: Jul. 16, 2026

(54) DENTAL COATING COMPOSITIONS AND METHODS OF MAKING THEREOF

(71) Applicants: Jonathan SEXSMITH, Stuart, FL (US); Whitney SEXSMITH, Stuart, FL (US)

(72) Inventors: Jonathan SEXSMITH, Stuart, FL (US); Whitney SEXSMITH, Stuart, FL (US)

(21) Appl. No.: 19/019,165

(22) Filed: Jan. 13, 2025

(51) Int. Cl. A61K 6/864; A61K 6/898

(52) U.S. Cl. CPC A61K 6/864; A61K 6/898

(57) ABSTRACT

Provided herein are dental coating compositions for providing long-term protection against tooth decay. The dental coating compositions can include nanohydroxyapatite, at least one fluoride-releasing agent, antimicrobial peptides, at least one bioadhesive polymer, and/or at least one pH buffering agent.

### FIELD

This disclosure relates generally to dental coating compositions and methods of making thereof. More specifically, this disclosure relates to dental coating compositions for long-term protection against tooth decay and methods of making thereof.

### BACKGROUND

Tooth decay is one of the most common oral health problems affecting populations worldwide. For example, acids can erode the enamel of teeth, which can lead to holes in the teeth known as dental caries or cavities. Dental caries can be painful and may lead to gum disease, facial swelling, bad breath, and/or sensitivity to hot or cold food and drinks.

Numerous preventative measures exist. For example, regular oral hygiene practices (e.g., brushing and flossing the teeth) can greatly reduce the incidence of tooth decay. Preventative treatments such as fluoride treatments and dental sealants can also be administered to patients. However, existing preventative treatments rely on strong patient compliance and proactivity. Even the longer-term solutions such as preventative fluoride and sealant treatments may require relatively frequent reapplication (e.g., every three to six months). Accordingly, poor patient compliance with regular dentist visits can limit the scope of protection provided by existing treatments.

### SUMMARY

Described herein are dental coating compositions for preventing tooth decay and methods of making and applying the same. The dental coating compositions described herein can be applied to the teeth of a patient to provide enhanced protection against several leading causes of tooth decay. The dental coating compositions can include nanohydroxyapatite for promoting remineralization and repair of microdefects in tooth enamel. The dental coating compositions can also include fluoride to enhance enamel strength and/or antimicrobial peptides that target cariogenic bacteria (e.g., Streptococcus mutans) to reduce plaque formation. The dental coating compositions can also include pH buffers to maintain a neutral oral environment, thus reducing the risk of enamel demineralization. The dental coating compositions disclosed herein can also include bioadhesive polymers to ensure durable adhesion of the dental coating compositions to tooth surfaces, thus improving the retention time of the dental coating compositions relative to existing preventative treatments.

The dental coating compositions described herein can provide several advantages over existing preventative treatments. For example, the bioadhesive polymer(s) in the dental coating compositions can provide improved adhesion to teeth compared to existing treatments. As a result, the frequency of reapplication may be reduced. Furthermore, the dental coating compositions may simultaneously provide antibacterial action through the inclusion of antimicrobial peptides, enamel strengthening from the nanohydroxyapatite and/or fluoride, and/or PH balance from the pH buffering agent(s). These components are known to be safe and well-tolerated in the oral environment. Moreover, the dental coating compositions may also be easily administered during routine dental visits without the need for specialized equipment.

An exemplary dental coating composition comprises: nanohydroxyapatite; at least one fluoride-releasing agent; at least one antimicrobial peptide; at least one bioadhesive polymer; and at least one pH buffering agent. The dental coating composition may include 1-30 wt. % the nanohydroxyapatite. The nanohydroxyapatite may have a D50 particle diameter of about 20-100 nm as measured by dynamic light scattering at 25° C. in an aqueous dispersion. The dental coating composition may include 0.1-5 wt. % the at least one fluoride-releasing agent. The at least one fluoride-releasing agent may include sodium fluoride, stannous fluoride, sodium monofluorophosphate, potassium fluoride, amine fluorides, or combinations thereof. The at least one fluoride-releasing agent may include at least one fluoride compound encapsulated within a biodegradable polymer. The dental coating composition may include polylactic-co-glycolic acid. The dental coating composition may include 0.01-1 wt. % the at least one antimicrobial peptide. The at least one antimicrobial peptide may include nisin, defensins, or combinations thereof. The at least one antimicrobial peptide may target at least one of Streptococcus mutans, Lactobacillus acidophilus, and Actinomyces viscosus. The dental coating composition may include 10-50 wt. % the at least one bioadhesive polymer. The at least one bioadhesive polymer may include chitosan, polyvinyl alcohol, polyacrylic acid, a methacrylate derivative, or combinations thereof. The dental coating composition may include 1-10 wt. % the at least one pH buffering agent. The at least one pH buffering agent may include calcium carbonate, sodium bicarbonate, a phosphate buffer, or combinations thereof. The pH buffering agent may be configured to maintain an oral pH of 6.5-7.5. The dental coating composition may include at least one flavoring agent. The at least one flavoring agent may include peppermint oil, spearmint oil, cinnamon oil, vanilla extract, citrus extracts, menthol, eucalyptol, or combinations thereof. The dental coating composition may include at least one coloring agent. The at least one coloring agent may include titanium dioxide, iron oxides, or combinations thereof. The dental coating composition may include at least one preservative. The at least one preservative may include potassium sorbate, phenoxyethanol, parabens, or combinations thereof. The dental coating composition may include at least one photoinitiator. The at least one photoinitiator may include camphorquinone, phenylpropanedione (PPD), trimethylbenzoyl diphenylphosphine oxide (TPO), phenylbis (2,4,6-trimethylbenzoyl)-phosphine oxide (BAPO), or combinations thereof. The dental coating composition may include at least one thickening agent. The at least one thickening agent may include carbomers, cellulose derivatives, fumed silica, or combinations thereof. The dental coating composition may include at least one plasticizer. The at least one plasticizer may include glycerol. The dental coating composition may include at least one solvent. The dental coating composition may include 10-60% the at least one solvent. The at least one solvent may include distilled water, ethanol, dichloromethane, ethyl acetate, or combinations thereof.

A viscosity of the dental coating composition may be about 1,000-5,000 cP. The dental coating composition may be configured to provide protection against tooth decay for at least six months. The dental coating composition may be configured to provide protection against tooth decay for at least twelve months. An adhesion strength of the dental coating composition to teeth may be about 5-15 MPa. The dental coating composition may be configured to release about 0.1-1 ppm fluoride per day into a mouth of a patient. Patients treated with the dental coating composition may experience about a 20-60% reduction in dental caries over a 12-month period.

An exemplary method of applying a dental coating composition to teeth of a patient comprises: coating the teeth with the dental coating composition; and curing the dental coating composition. Coating the teeth with the dental coating composition may include applying the dental coating composition to the teeth with a microbrush, an applicator tip, or a spray device. Curing the dental coating composition may include allowing the dental coating composition to set for a predetermined amount of time at room temperature. Curing the dental coating composition may include exposing the dental coating composition to a curing light. The curing light may include an LED light, a halogen light, a UV light, or combinations thereof. Prior to coating the teeth with the dental coating composition, plaque and/or tartar may be removed from the teeth. Prior to coating the teeth with the dental coating composition, an etching solution may be applied to the teeth. The etching solution may include 10% phosphoric acid. The etching solution may be applied for 15-30 seconds.

An exemplary method of preparing a dental coating composition comprises: dissolving at least one bioadhesive polymer in a solvent to form a solution; adding nanohydroxyapatite, at least one fluoride-releasing agent, antimicrobial peptides, and at least one pH buffering agent to the solution; and mixing the solution to form the dental coating composition. The dental coating composition may be a dental coating composition described herein. The solvent may include distilled water, ethanol, or combinations thereof. Prior to adding the at least one fluoride-releasing agent to the solution, at least one fluoride compound may be encapsulated in a biodegradable polymer to form the at least one fluoride-releasing agent. The method may further include adding at least one thickening agent to the solution. The method may further include sterilizing the dental coating composition. Sterilizing the dental coating composition may include irradiating the dental coating composition with gamma radiation, autoclaving the dental coating composition, or performing aseptic processing.

It will be appreciated that any of the variations, aspects, features and options described in view of the systems, methods, and/or compositions apply equally to the systems, methods, and/or compositions, and vice versa. It will also be clear that any one or more of the above variations, aspects, embodiments, features, and options can be combined.

Additional advantages will be readily apparent to those skilled in the art from the following detailed description. The examples and descriptions herein are to be regarded as illustrative in nature and not restrictive.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE FIGURES

The invention will now be described, by way of example only, with reference to the accompanying drawings, in which:

FIG. 1 illustrates an exemplary method of making a dental coating composition in accordance with some embodiments disclosed herein.

FIG. 2 illustrates an exemplary method of applying a dental coating composition in accordance with some embodiments disclosed herein.

### DETAILED DESCRIPTION

Reference will now be made in detail to implementations and embodiments of various aspects and variations of compositions and methods disclosed herein, examples of which are provided herein. Various compositions and methods are described herein. Although several exemplary variations of the compositions and methods are described, other variations may include aspects or embodiments of the compositions and methods described herein combined in any suitable manner having combinations of all or some of the aspects or embodiments described. Exemplary aspects and embodiments will now be described more fully hereinafter; however, they may be embodied in different forms and should not be construed as limited to the examples set forth herein. Rather, these examples are provided so that this disclosure will be thorough and complete, and will fully convey exemplary implementations to those skilled in the art.

Described herein are dental coating compositions and methods for preparing and applying the same. The dental coating compositions can include nanohydroxyapatite, at least one fluoride-releasing agent, at least one antimicrobial peptide, at least one bioadhesive polymer, and at least one pH buffering agent. When applied to the teeth of a patient, the dental coating compositions can provide long-term protection against tooth decay by promoting remineralization of enamel (and dentin) and/or maintaining a neutral oral pH to prevent so-called “acid attacks,” which can occur when bacteria in the oral environment produces acids that attack enamel.

A dental coating composition disclosed herein may include nanohydroxyapatite. Nanohydroxyapatite is a synthetic form of hydroxyapatite, which is a mineral that is a main component of enamel and bone. Nanohydroxyapatite can mimic natural enamel and can promote remineralization of enamel. Due to its small size, nanohydroxyapatite particles (or powder) can penetrate into the pores of the enamel and repair microdefects, thus preventing dental caries from forming. In some embodiments, the nanohydroxyapatite in the dental coating composition has a D50 particle diameter that may be at least about 20 nm, at least about 30 nm, at least about 40 nm, at least about 50 nm, at least about 60 nm, at least about 70 nm, at least about 80 nm, or at least about 90 nm. In some embodiments, the nanohydroxyapatite in the dental coating composition has a D50 particle diameter that may be at most about 100 nm, at most about 90 nm, at most about 80 nm, at most about 70 nm, at most about 60 nm, at most about 50 nm, at most about 40 nm, or at most about 30 nm. In some embodiments, the nanohydroxyapatite in the dental coating composition has a D50 particle diameter that may be about 20-100 nm or about 40-80 nm, as measured by dynamic light scattering at 25° C. in an aqueous dispersion and/or transmission electron microscopy.

In some embodiments, the dental coating composition may include about 1-30% nanohydroxyapatite by weight, about 10-20% nanohydroxyapatite by weight, or about 15% nanohydroxyapatite by weight. In some embodiments, the dental coating composition may include at least about 1% nanohydroxyapatite by weight, at least about 5% nanohydroxyapatite by weight, at least about 10% nanohydroxyapatite by weight, at least about 15% nanohydroxyapatite by weight, at least about 20% nanohydroxyapatite by weight, or at least about 25% nanohydroxyapatite by weight. In some embodiments, the dental coating composition may include at most about 30% nanohydroxyapatite by weight, at most about 25% nanohydroxyapatite by weight, at most about 20% nanohydroxyapatite by weight, at most about 15% nanohydroxyapatite by weight, at most about 10% nanohydroxyapatite by weight, or at most about 5% nanohydroxyapatite by weight.

The dental coating composition may also include at least one fluoride-releasing agent. When the dental coating composition is applied to the teeth of a patient, the fluoride-releasing agent(s) may release fluoride ions into the oral environment to strengthen enamel and/or promote resistance to harmful acids. In some embodiments, the fluoride-releasing agents may include one or more fluoride compounds, such as sodium fluoride, stannous fluoride, sodium monofluorophosphate, potassium fluoride, amine fluorides (e.g., Olaflur), acidulated phosphate fluoride, and/or calcium fluoride. In some embodiments, the fluoride-releasing agent can be an encapsulated fluoride compound. In some embodiments, the one or more fluoride compounds may be encapsulated within biodegradable polymers. Encapsulating a fluoride compound within a biodegradable polymer may prolong the time horizon over which fluoride ions are released from the dental coating composition. In some examples, a fluoride-releasing agent may include one or more fluoride compounds encapsulated within polylactic-co-glycolic acid (PLGA) (i.e., a biodegradable polymer).

In some embodiments, the dental coating composition may include about 0.1-5% fluoride-releasing agent by weight or about 1-3% fluoride-releasing agent by weight. In some embodiments, the dental coating composition may include at least about 0.1% fluoride-releasing agent by weight, at least about 0.5% fluoride-releasing agent by weight, at least about 1% fluoride-releasing agent by weight, at least about 1.5% fluoride-releasing agent by weight, at least about 2% fluoride-releasing agent by weight, at least about 2.5% fluoride-releasing agent by weight, at least about 3% fluoride-releasing agent by weight, at least about 3.5% fluoride-releasing agent by weight, at least about 4% fluoride-releasing agent by weight, or at least about 4.5% fluoride-releasing agent by weight. In some embodiments, the dental coating composition may include at most about 5% fluoride-releasing agent by weight, at most about 4.5% fluoride-releasing agent by weight, at most about 4% fluoride-releasing agent by weight, at most about 3.5% fluoride-releasing agent by weight, at most about 3% fluoride-releasing agent by weight, at most about 2.5% fluoride-releasing agent by weight, at most about 2% fluoride-releasing agent by weight, at most about 1.5% fluoride-releasing agent by weight, at most about 1% fluoride-releasing agent by weight, or at most about 0.5% fluoride-releasing agent by weight.

In some embodiments, the dental coating composition may also include at least one antimicrobial peptide. In some embodiments, antimicrobial peptides may be naturally occurring and/or synthetic peptides that have inhibitory effects against bacteria. In some embodiments, the antimicrobial peptides used in the dental coating composition may target cariogenic bacteria (e.g., Streptococcus mutans). By killing cariogenic bacteria, the antimicrobial peptides can reduce plaque formation, thus reducing the buildup of acid-producing bacteria. In some embodiments, the antimicrobial peptides may include nisin, defensins, and/or custom-designed antimicrobial peptides effective against Streptococcus mutans, Lactobacillus acidophilus, and/or Actinomyces viscosus. In some embodiments, the antimicrobial peptides may be encapsulated using nanoencapsulation techniques. The antimicrobial peptides may be encapsulated in PLGA, chitosan, alginate, liposomes, or cyclodextrins.

In some embodiments, the dental coating composition may include about 0.01-1%, about 0.05-0.5%, or 0.1-0.2% at least one antimicrobial peptide by weight. In some embodiments, the dental coating composition may include at least about 0.01% antimicrobial peptides by weight, at least about 0.025% antimicrobial peptides by weight, at least about 0.05% antimicrobial peptides by weight, at least about 0.075% antimicrobial peptides by weight, at least about 0.1% antimicrobial peptides by weight, at least about 0.25% antimicrobial peptides by weight, at least about 0.5% antimicrobial peptides by weight, or at least about 0.75% antimicrobial peptides by weight. In some embodiments, the dental coating composition may include at most about 1% antimicrobial peptides by weight, at most about 0.75% antimicrobial peptides by weight, at most about 0.5% antimicrobial peptides by weight, at most about 0.25% antimicrobial peptides by weight, at most about 0.1% antimicrobial peptides by weight, at most about 0.075% antimicrobial peptides by weight, at most about 0.05% antimicrobial peptides by weight, or at most about 0.025% antimicrobial peptides by weight.

In some embodiments, the dental coating composition may further include at least one bioadhesive polymer. Bioadhesive polymers may provide strong adhesion of the dental coating composition to enamel and/or dentin of the teeth, ensuring durability of the coating. Suitable bioadhesive polymers may include chitosan, polyvinyl alcohol, polyacrylic acid, and/or methacrylate derivatives (e.g., Bis-GMA, UDMA, TEGDMA, MMA, Bis-EMA, or a combination thereof).

In some embodiments, the dental coating composition may include about 10-50% bioadhesive polymer by weight, about 20-40% bioadhesive polymer by weight, or about 25-30% bioadhesive polymer by weight. In some embodiments, the dental coating composition may include at least about 10% bioadhesive polymer by weight, at least about 15% bioadhesive polymer by weight, at least about 20% bioadhesive polymer by weight, at least about 25% bioadhesive polymer by weight, at least about 30% bioadhesive polymer by weight, at least about 35% bioadhesive polymer by weight, at least about 40% bioadhesive polymer by weight, or at least about 45% bioadhesive polymer by weight. In some embodiments, the dental coating composition may include at most about 50% bioadhesive polymer by weight, at most about 45% bioadhesive polymer by weight, at most about 40% bioadhesive polymer by weight, at most about 35% bioadhesive polymer by weight, at most about 30% bioadhesive polymer by weight, at most about 25% bioadhesive polymer by weight, at most about 20% bioadhesive polymer by weight, or at most about 15% bioadhesive polymer by weight.

In some embodiments, the dental coating composition may also include at least one pH buffering agent. Tooth decay can be caused by buildup of bacteria that release acid, which can break down enamel. The pH buffering agent(s) may help to maintain a substantially neutral oral environment. In some embodiments, the pH buffering agent(s) may maintain the pH of the oral environment at about 5-8 or about 6.5-7.5. In some embodiments, the pH buffering agent(s) may maintain the pH of the oral environment at a pH of at least about 6.5, a pH of at least about 6.75, a pH of at least about 7.0, or a pH of at least about 7.25. In some examples, the pH buffering agent(s) may maintain the pH of the oral environment at a pH of at most about 8, at most about 7.5, at most about 7.25, at most about 7.0, or at most about 6.75. Suitable pH buffering agents may include calcium carbonate, sodium bicarbonate, and/or phosphate buffers.

In some embodiments, the dental coating composition may include about 1-10% pH buffering agent by weight or about 2-5% pH buffering agent by weight. In some examples, the dental coating composition may include at least about 1% pH buffering agent by weight, at least about 2% pH buffering agent by weight, at least about 3% pH buffering agent by weight, at least about 4% pH buffering agent by weight, at least about 5% pH buffering agent by weight, at least about 6% pH buffering agent by weight, at least about 7% pH buffering agent by weight, at least about 8% pH buffering agent by weight, or at least about 9% pH buffering agent by weight. In some examples, the dental coating composition may include at most about 10% pH buffering agent by weight, at most about 9% pH buffering agent by weight, at most about 8% pH buffering agent by weight, at most about 7% pH buffering agent by weight, at most about 6% pH buffering agent by weight, at most about 5% pH buffering agent by weight, at most about 4% pH buffering agent by weight, at most about 3% pH buffering agent by weight, or at most about 2% pH buffering agent by weight.

In some embodiments, the dental coating composition may include one or more flavoring agents. Flavoring agents may contribute to improved patient compliance. Suitable flavoring agents may include peppermint oil, spearmint oil, cinnamon oil, vanilla extract, citrus extracts, menthol, and/or eucalyptol. The dental coating composition may include about 0.01-5% flavoring agent by weight, about 0.05-3% flavoring agent by weight, about 0.1-2% flavoring agent by weight, or about 0.1-0.5% flavoring agent by weight. In some embodiments, the dental coating composition may include at least about 0.01% flavoring agent by weight, at least about 0.05% flavoring agent by weight, at least about 0.1% flavoring agent by weight, at least about 0.5% flavoring agent by weight, at least about 1% flavoring agent by weight, at least about 1.5% flavoring agent by weight, at least about 2% flavoring agent by weight, or at least about 2.5% flavoring agent by weight. In some embodiments, the dental coating composition may include at most about 5% flavoring agent by weight, at most about 3% flavoring agent by weight, at most about 2.5% flavoring agent by weight, at most about 2% flavoring agent by weight, at most about 1.5% flavoring agent by weight, at most about 1% flavoring agent by weight, at most about 0.5% flavoring agent by weight, at most about 0.1% flavoring agent by weight, or at most about 0.05% flavoring agent by weight.

In some embodiments, the dental coating composition may include one or more coloring agents. Coloring agents may be added for aesthetic purposes. For example, titanium dioxide may be added for whiteness and/or opacification. Iron oxides and/or other

FDA-approved dyes may be added to provide subtle coloration (e.g., to match a patient's natural tooth color). The dental coating composition may include about 0.1-2% coloring agent by weight or about 0.5-1% coloring agent by weight. In some embodiments, the dental coating composition may include at least about 0.1% coloring agent by weight, at least about 0.25% coloring agent by weight, at least about 0.5% coloring agent by weight, at least about 0.75% coloring agent by weight, at least about 1% coloring agent by weight, at least about 1.25% coloring agent by weight, at least about 1.5% coloring agent by weight, or at least about 1.75% coloring agent by weight. In some embodiments, the dental coating composition may include at most about 2% coloring agent by weight, at most about 1.75% coloring agent by weight, at most about 1.5% coloring agent by weight, at most about 1.25% coloring agent by weight, at most about 1% coloring agent by weight, at most about 0.75% coloring agent by weight, at most about 0.5% coloring agent by weight, or at most about 0.25% coloring agent by weight.

In some embodiments, the dental coating composition may include one or more preservatives. Preservatives may prolong the shelf life of the dental coating composition, for example to about 12 to 36 months or about 12-24 months at room temperature. This is a significant improvement over existing dental coating compositions, which have a shelf life of about 3-6 months at room temperature. Suitable preservatives may include potassium sorbate, phenoxyethanol, and/or parabens. The dental coating composition may include about 0.1-1% preservatives. In some embodiments, the dental coating composition may include at least about 0.1% preservative by weight, at least about 0.2% preservative by weight, at least about 0.3% preservative by weight, at least about 0.4% preservative by weight, at least about 0.5% preservative by weight, at least about 0.6% preservative by weight, at least about 0.7% preservative by weight, at least about 0.8% preservative by weight, or at least about 0.9% preservative by weight. In some embodiments, the dental coating composition may include at most about 1% preservative by weight, at most about 0.9% preservative by weight, at most about 0.8% preservative by weight, at most about 0.7% preservative by weight, at most about 0.6% preservative by weight, at most about 0.5% preservative by weight, at most about 0.4% preservative by weight, at most about 0.3% preservative by weight, or at most about 0.2% preservative by weight.

In some embodiments, the dental coating composition may include one or more photoinitiators. The one or more photoinitiators may be used to cure the dental coating composition once the dental coating composition has been applied to the teeth of a patient. For example, a photoinitiator present in the dental coating composition may be activated by ultraviolet (UV) radiation. The dental coating composition may be applied to the teeth of a patient, and the teeth may be exposed to UV radiation for a predetermined amount of time. The photoinitiator may absorb the UV radiation and responsively form reactive species. The reactive species may then initiate reactions (e.g., polymerization chain reactions) that cause the dental coating composition to harden and adhere to the teeth. In some examples, suitable photoinitiators may include camphorquinone, phenylpropanedione (PPD), trimethylbenzoyl diphenylphosphine oxide (TPO), and/or phenylbis(2,4,6-trimethylbenzoyl)-phosphine oxide (BAPO). In some embodiments, the dental coating composition may further include one or more co-initiators, such as an amine co-initiator.

The dental coating composition may include about 0.1-2% photoinitiator by weight. The photoinitiator(s) may be added to the dental coating composition using precise gravimetric addition, and the presence of the photoinitiator(s) may be confirmed using Fourier Transform Infrared (FTIR) spectroscopy and/or UV-Vis spectral analysis. In some embodiments, the dental coating composition may include at least about 0.1% photoinitiator by weight, at least about 0.25% photoinitiator by weight, at least about 0.5% photoinitiator by weight, at least about 0.75% photoinitiator by weight, at least about 1% photoinitiator by weight, at least about 1.25% photoinitiator by weight, at least about 1.5% photoinitiator by weight, or at least about 1.75% photoinitiator by weight. In some embodiments, the dental coating composition may include at most about 2% photoinitiator by weight, at most about 1.75% photoinitiator by weight, at most about 1.5% photoinitiator by weight, at most about 1.25% photoinitiator by weight, at most about 1% photoinitiator by weight, at most about 0.75% photoinitiator by weight, at most about 0.5% photoinitiator by weight, or at most about 0.25% photoinitiator by weight.

In some embodiments, the dental coating composition may include one or more plasticizers. The one or more plasticizers may include glycerol, polyethylene glycol, triethyl citrate, or any other suitable plasticizers. The dental coating composition may include about 5-25% plasticizer by weight, about 10-20% plasticizer by weight, or about 12.5-17.5% plasticizer by weight. In some embodiments, the dental coating composition may include at least about 5% plasticizer by weight, at least about 7.5% plasticizer by weight, at least about 10% plasticizer by weight, at least about 12.5% plasticizer by weight, at least about 15% plasticizer by weight, at least about 17.5% plasticizer by weight, at least about 20% plasticizer by weight, or at least about 22.5% plasticizer by weight. In some embodiments, the dental coating composition may include at most about 25% plasticizer by weight, at most about 22.5% plasticizer by weight, at most about 20% plasticizer by weight, at most about 17.5% plasticizer by weight, at most about 15% plasticizer by weight, at most about 12.5% plasticizer by weight, at most about 10% plasticizer by weight, or at most about 7.5% plasticizer by weight.

In some embodiments, the dental coating composition may include one or more thickening agents. The thickening agent(s) may be used to fine-tune the viscosity of the dental coating composition (e.g., to achieve a viscosity of about 1,000-5,000 cP). Suitable thickening agents may include carbomers, cellulose derivatives, and/or fumed silica. The dental coating composition may include about 0.01-5% or about 0.1-3% thickening agent by weight. In some embodiments, the dental coating composition may include at least about 0.01% thickening agent by weight, at least about 0.05% thickening agent by weight, at least about 0.1% thickening agent by weight, at least about 0.5% thickening agent by weight, at least about 1% thickening agent by weight, at least about 1.5% thickening agent by weight, at least about 2% thickening agent by weight, at least about 2.5% thickening agent by weight, at least about 3% thickening agent by weight, at least about 3.5% thickening agent by weight, at least about 4% thickening agent by weight, or at least about 4.5% thickening agent by weight. In some embodiments, the dental coating composition may include at most about 5% thickening agent by weight, at most about 4.5% thickening agent by weight, at most about 4% thickening agent by weight, at most about 3.5% thickening agent by weight, at most about 3% thickening agent by weight, at most about 2.5% thickening agent by weight, at most about 2% thickening agent by weight, at most about 1.5% thickening agent by weight, at most about 1% thickening agent by weight, at most about 0.5% thickening agent by weight, at most about 0.1% thickening agent by weight, or at most about 0.05% thickening agent by weight.

In some embodiments, the dental coating composition may include one or more solvents. The one or more solvents may be used to dissolve one or more other components of the dental coating composition during preparation of the dental coating composition. The one or more solvents may include distilled water, ethanol, dichloromethane, ethyl acetate, or combinations thereof. In some embodiments, the dental coating composition may include about 10-60% solvent by weight, about 20-50% solvent by weight, or about 30-40% solvent by weight. In some embodiments, the dental coating composition may include at least about 10% solvent by weight, at least about 15% solvent by weight, at least about 20% solvent by weight, at least about 30% solvent by weight, at least about 35% solvent by weight, at least about 40% solvent by weight, at least about 45% solvent by weight, at least about 50% solvent by weight, or at least about 55% solvent by weight. In some embodiments, the dental coating composition may include at most about 60% solvent by weight, at most about 55% solvent by weight, at most about 50% solvent by weight, at most about 45% solvent by weight, at most about 40% solvent by weight, at most about 35% solvent by weight, at most about 30% solvent by weight, or at most about 25% solvent by weight. In some embodiments, the dental coating composition may include multiple solvents. For example, the dental coating composition may include both distilled water and ethanol. In some embodiments, the dental coating composition may include about 1-20% ethanol by weight, about 5-15% ethanol by weight, or about 10% ethanol by weight. In some embodiments, the dental coating composition may include at least about 1% ethanol by weight, at least about 5% ethanol by weight, at least about 10% ethanol by weight, or at least about 15% ethanol by weight. In some embodiments, the dental coating composition may include at most about 20% ethanol by weight, at most about 15% ethanol by weight, or at most about 10% ethanol by weight. In some embodiments, the dental coating composition may include about 10-50% distilled water by weight, about 20-40% distilled water by weight, or about 30% distilled water by weight. In some embodiments, the dental coating composition may include at least about 10% distilled water by weight, at least about 15% distilled water by weight, at least about 20% distilled water by weight, at least about 25% distilled water by weight, at least about 30% distilled water by weight, at least about 35% distilled water by weight, at least about 40% distilled water by weight, or at least about 45% distilled water by weight. In some embodiments, the dental coating composition may include at most about 50% distilled water by weight, at most about 45% distilled water by weight, at most about 40% distilled water by weight, at most about 35% distilled water by weight, at most about 30% distilled water by weight, or at most about 25% distilled water by weight.

The dental coating composition may be applied to the teeth of a patient. In some embodiments, the dental coating composition may be configured to provide long-term protection against tooth decay by releasing fluoride and/or antimicrobial peptides into the mouth of the patient, maintaining a neutral oral pH to protect against acid attacks, and/or promoting remineralization of enamel and/or dentin. In some embodiments, the dental coating composition may be configured to provide protection against tooth decay for at least about six months, at least about twelve months, at least about eighteen months, or at least about twenty-four months. In some embodiments, the dental coating composition may be configured to provide protection against tooth decay for at most about thirty months, at most about twenty-four months, at most about eighteen months, or at most about twelve months. In some embodiments, the dental coating composition may be configured to provide protection against tooth decay for six months up to several years.

Various performance characteristics of the dental coating composition can be measured. Adhesion strength of the dental coating composition to teeth can be measured using an in-vitro shear bond test on bovine enamel. In some embodiments, the adhesion strength can be about 5-15 MPa or about 8-12 MPa. In some embodiments, adhesion strength of the dental coating composition to teeth may be at least about 5 MPa, at least about 6 MPa, at least about 7 MPa, at least about 8 MPa, at least about 9 MPa, at least about 10 MPa, at least about 11 MPa, at least about 12 MPa, at least about 13 MPa, or at least about 14 MPa. In some embodiments, adhesion strength of the dental coating composition to teeth may be at most about 15 MPa, at most about 14 MPa, at most about 13 MPa, at most about 12 MPa, at most about 11 MPa, at most about 10 MPa, at most about 9 MPa, at most about 8 MPa, at most about 7 MPa, or at most about 6 MPa.

The release of fluoride from the dental coating composition can be measured in simulated salivary conditions (pH 6.8). The dental coating composition can release about 0.1-1 ppm fluoride per day or about 0.3-0.5 ppm fluoride per day over a fourteen day period in artificial saliva. In some embodiments, the dental coating composition may release at least about 0.1 ppm fluoride per day, at least about 0.2 ppm fluoride per day, at least about 0.3 ppm fluoride per day, at least about 0.4 ppm fluoride per day, at least about 0.5 ppm fluoride per day, at least about 0.6 ppm fluoride per day, at least about 0.7 ppm fluoride per day, at least about 0.8 ppm fluoride per day, or at least about 0.9 ppm fluoride per day. In some embodiments, the dental coating composition may release at most about 1 ppm fluoride per day, at most about 0.9 ppm fluoride per day, at most about 0.8 ppm fluoride per day, at most about 0.7 ppm fluoride per day, at most about 0.6 ppm fluoride per day, at most about 0.5 ppm fluoride per day, at most about 0.4 ppm fluoride per day, at most about 0.3 ppm fluoride per day, or at most about 0.2 ppm fluoride per day.

Antimicrobial efficacy can also be measured using zone-of-inhibition assays. About a 50-99% reduction, about a 75-95% reduction or about a 90% reduction in Streptococcus mutans can be observed within 24 hours. In some embodiments, at least a 50% reduction in Streptococcus mutans may be observed within 24 hours, at least a 55% reduction in Streptococcus mutans may be observed within 24 hours, at least a 60% reduction in Streptococcus mutans may be observed within 24 hours, at least a 65% reduction in Streptococcus mutans may be observed within 24 hours, at least a 70% reduction in Streptococcus mutans may be observed within 24 hours, at least a 75% reduction in Streptococcus mutans may be observed within 24 hours, at least an 80% reduction in Streptococcus mutans may be observed within 24 hours, at least an 85% reduction in Streptococcus mutans may be observed within 24 hours, at least a 90% reduction in Streptococcus mutans may be observed within 24 hours, or at least a 95% reduction in Streptococcus mutans may be observed within 24 hours. In some embodiments, at most a 99% reduction in Streptococcus mutans may be observed within 24 hours, at most a 95% reduction in Streptococcus mutans may be observed within 24 hours, at most a 90% reduction in Streptococcus mutans may be observed within 24 hours, at most an 85% reduction in Streptococcus mutans may be observed within 24 hours, at most an 80% reduction in Streptococcus mutans may be observed within 24 hours, at most a 75% reduction in Streptococcus mutans may be observed within 24 hours, at most a 70% reduction in Streptococcus mutans may be observed within 24 hours, at most a 65% reduction in Streptococcus mutans may be observed within 24 hours, at most a 60% reduction in Streptococcus mutans may be observed within 24 hours, or at most a 55% reduction in Streptococcus mutans may be observed within 24 hours.

In vivo trials can demonstrate that patients whose teeth were treated with the dental coating composition experience about a 20-60% reduction, about a 30-50% reduction, or about a 35% reduction in dental caries over a 12-month period. In some embodiments, patients may experience at least about a 20% reduction in dental caries over a 12-month period, at least about a 25% reduction in dental caries over a 12-month period, at least about a 30% reduction in dental caries over a 12-month period, at least about a 35% reduction in dental caries over a 12-month period, at least about a 40% reduction in dental caries over a 12-month period, at least about a 45% reduction in dental caries over a 12-month period, at least about a 50% reduction in dental caries over a 12-month period, or at least about a 55% reduction in dental caries over a 12-month period. In some embodiments, patients may experience at most about a 60% reduction in dental caries over a 12-month period, at most about a 55% reduction in dental caries over a 12-month period, at most about a 50% reduction in dental caries over a 12-month period, at most about a 45% reduction in dental caries over a 12-month period, at most about a 40% reduction in dental caries over a 12-month period, at most about a 35% reduction in dental caries over a 12-month period, at most about a 30% reduction in dental caries over a 12-month period, or at most about a 25% reduction in dental caries over a 12-month period.

### Methods of Preparation

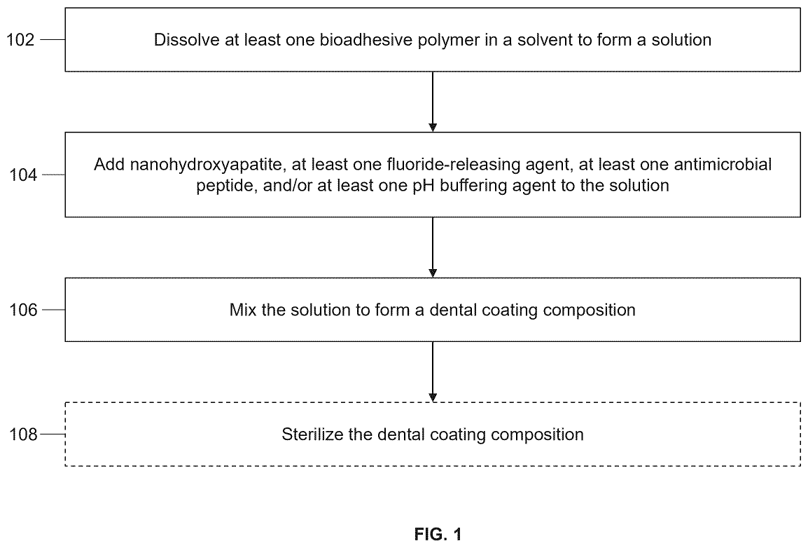

FIG. 1 illustrates an exemplary method 100 for preparing the dental coating compositions described herein. According to some aspects, techniques other than those described herein may be used to prepare the dental coating compositions.

At step 102, at least one bioadhesive polymer can be dissolved in a solvent to form a solution. The at least one bioadhesive polymer may be chitosan, polyvinyl alcohol, polyacrylic acid, and/or a methacrylate derivative. The solvent may be distilled water, ethanol, and/or any other appropriate solvent, or combinations thereof. The ratio of bioadhesive polymer to solvent by weight may be about 1:5 to about 1:10. In some embodiments, a mild acid (e.g., acetic acid) may be added to the solution to facilitate polymer solubilization. The pH may be readjusted back to the initial pH of the solution once the polymer has completely dissolved.

At step 104, nanohydroxyapatite, at least one fluoride-releasing agent, at least one antimicrobial peptide, and/or at least one pH buffering agent are added to the solution. In some embodiments, the solution can be added to the nanohydroxyapatite, at least one fluoride-releasing agent, at least one antimicrobial peptide, and/or at least one pH buffering agent. Upon addition of these components, the solution may include about 1-30% nanohydroxyapatite by weight, about 0.1-5% fluoride-releasing agent by weight, about 0.01-1% antimicrobial peptides by weight, about 10-50% bioadhesive polymer by weight, and about 1-10% pH buffering agent by weight. As discussed above, the at least one fluoride-releasing agent may include sodium fluoride, sodium monofluorophosphate, potassium fluoride, amine fluorides, and/or stannous fluoride optionally encapsulated in a biodegradable polymer (e.g., PLGA). The antimicrobial peptides may include nisin, defensins, and/or custom-designed peptides effective against Streptococcus mutans and may be encapsulated (e.g., in PLGA, chitosan, alginate, liposomes, or cyclodextrins). The at least one pH buffering agent may include calcium carbonate, sodium bicarbonate, and/or phosphate buffers. One or more thickening agents (e.g., carbomers or cellulose derivatives), flavoring agents (e.g., peppermint oil, spearmint oil, cinnamon oil, vanilla extract, citrus extracts, menthol, and/or eucalyptol), coloring agents (e.g., titanium dioxide and/or iron oxides), preservatives (e.g., potassium sorbate, phenoxyethanol, and/or parabens), photoinitiators (e.g., camphorquinone, PPD, TPO, and/or BAPO), and/or plasticizers (e.g., glycerol) may also be added to the solution.

Pre-formulated or existing nanohydroxyapatite may be obtained or used, or nanohydroxyapatite may be synthesized prior to performing step 104. In some embodiments, nanohydroxyapatite may be synthesized using wet chemical precipitation techniques. For example, calcium nitrate (e.g., calcium nitrate tetrahydrate) may be dissolved in deionized water (in a first vessel). Diammonium hydrogen phosphate may also be dissolved in deionized water (in a second vessel). The diammonium hydrogen phosphate solution may be slowly added to the calcium nitrate solution while stirring constantly. In some embodiments, the pH of the mixture may be adjusted to about 10 using ammonium hydroxide. In some embodiments, the reaction may be carried out at ambient conditions. In some embodiments, the temperature may be adjusted to control the size of the nanohydroxyapatite particles/powder. For example, the reaction may be carried out at an elevated temperature (e.g., about 60-80° C.) in order to yield nanohydroxyapatite particles having a D50 diameter of about 40-80 nm. The reaction temperature may be kept below about 90° C. so as to avoid aggregation or sintering. The mixture may then be centrifuged at about 8,000-12,000 rpm for about 10-30 minutes. The mixture may be centrifuged multiple times, for example about 2-3 times. The precipitate may be collected (e.g., via centrifugation), washed, and/or dried. The nanohydroxyapatite can then be added to the mixture at step 104 in an appropriate amount.

As previously discussed, the fluoride-releasing agent may include a fluoride compound (e.g., sodium fluoride, sodium monophosphate, potassium fluoride, amine fluorides, acidulated phosphate fluoride, calcium fluoride, and/or stannous fluoride) encapsulated within a biodegradable polymer (e.g., PLGA). In some embodiments, the fluoride compound may be encapsulated prior to performing step 104. In some embodiments, the fluoride compound may be dissolved in distilled water, and the biodegradable polymer may be dissolved in a suitable solvent, such as dichloromethane or ethyl acetate, to form a solution. The ratio of biodegradable polymer to solvent by weight may be about 1:5 to about 1:10. The dissolved fluoride compound may be added to the solution such that the ratio of fluoride to polymer by weight in the solution is about 1:4 to about 1:20. In some embodiments, microencapsulation techniques (e.g., solvent evaporation and/or spray drying) may be used to form microspheres with the solution. For example, the solvent may be removed by evaporation or via spray drying. Other microencapsulation techniques may be used.

In some embodiments, antimicrobial peptides may be encapsulated prior to performing step 104. The antimicrobial peptides may include nisin, defensins, and/or custom-designed antimicrobial peptides effective against Streptococcus mutans, Lactobacillus acidophilus, and/or Actinomyces viscosus. The antimicrobial peptides may be encapsulated within, for example, PLGA, chitosan, or alginate using nanoencapsulation techniques. In some embodiments, the at least one antimicrobial peptide can be incorporated into the polymer matrix via mixing at step 104.

At step 106, the solution can be mixed to form a dental coating composition. In some embodiments, the solution may be mixed by stirring vigorously to ensure a uniform distribution of the nanohydroxyapatite, fluoride-releasing agent(s), antimicrobial peptide(s), pH buffering agent(s), and/or bioadhesive polymer(s) within the solution. In some embodiments, the solution may be mechanically stirred at about 200-500 rpm for about 10-30 minutes. Alternatively, the solution may be mixed using gentle ultrasonication to minimize agglomeration.

In some embodiments, the dental coating composition may be modified after mixing in order to achieve desired properties. For example, additional bioadhesive polymer may be added in order to increase the concentration of the bioadhesive polymer in the dental coating composition. Alternatively or in addition, one or more thickening agents may be added to the dental coating composition to increase the viscosity. Increasing the viscosity of the dental coating composition may improve adhesion of the dental coating composition to the teeth of a patient and/or may facilitate application of the dental coating composition to the teeth. For example, the viscosity of the dental coating composition may need to be within a predetermined range in order to be applied to the teeth using certain application instruments, such as sprayers or brushes (e.g., about 1,000-5,000 cP or about 2,500-3,000 cP at 25° C.). In some examples, suitable thickening agents may include carbomers or cellulose derivatives. In some embodiments, the pH of the dental coating composition may need to be within a predetermined range (e.g., about 6.5-7.5). Phosphate buffer or a mild acid or base may be added as needed to bring the pH into the desired range. In some embodiments, flavoring agent(s) may be added in order to achieve a desired taste, and/or coloring agent(s) may be added to achieve a desired appearance (e.g., off-white and slightly opaque). In some embodiments, plasticizer(s) may be added in order to adjust plasticity of the dental coating composition.

Optionally, at step 108, the dental coating composition may be sterilized. The dental coating composition may be sterilized by gamma irradiation techniques (e.g., by irradiating the dental coating composition at a dosage of about 25-30 kGy). Gamma irradiation techniques can utilize gamma rays to kill or inactivate microorganisms. Gamma irradiation may be used for sterilization if the dental coating composition is sensitive to heat, since gamma irradiation is not reliant on high temperatures. If the dental coating composition is heat-stable (e.g., within the range of about 200-300° C.), the dental coating composition may be sterilized by autoclaving. Alternatively, the dental coating composition may be sterilized by aseptic processing, in which the dental coating composition and the container in which it will be packaged are separately sterilized (e.g., using heat-based treatments or chemical sterilants), and sterility is maintained during packaging.

The dental coating composition may be stored in a sealed, light-proof container at about 4-25° C. In some embodiments, the shelf life of the dental coating composition at room temperature (about 25° C.) is about 12-36 months, about 12-24 months, or about 12-18 months. In some embodiments, the shelf life of the dental coating composition at room temperature is at least about 12 months, at least about 18 months, at least about 24 months, or at least about 30 months. In some embodiments, the shelf life of the dental coating composition at room temperature is at most about 36 months, at most about 30 months, at most about 24 months, or at most about 18 months.

### Methods of Application

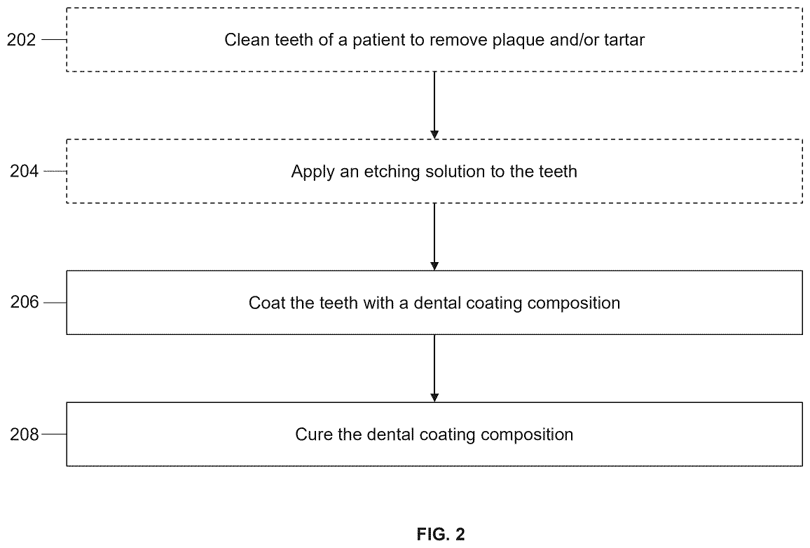

FIG. 2 illustrates an exemplary method 200 for applying a dental coating composition to teeth of a patient. According to some aspects, techniques other than those described herein may be used to apply dental coating composition.

At optional step 202, teeth of a patient can be cleaned to remove plaque and/or tartar. Removing plaque and/or tartar from the teeth may improve adhesion of a dental coating composition to the teeth. In some embodiments, the patient's teeth may be cleaned professionally, such as by a dentist or other dental professional.

At optional step 204, an etching solution can be applied to the teeth. The etching solution may dissolve at least some of the enamel and/or dentin of the teeth so as to create a rougher surface with microretentive features (e.g., with small fissures and/or ridges). The presence of the fissures and/or ridges may improve bonding of a dental coating composition to the teeth. In some embodiments, the etching solution may be a mild etching solution, such as 10% phosphoric acid. The etching solution may be applied for a predetermined period of time (e.g., about 15-30 seconds). The etching solution may be rinsed off the teeth with water, and the teeth may be subsequently dried.

At step 206, the teeth can be coated with a dental coating composition. The dental coating composition may be a dental coating composition described herein. The dental coating composition may be coated onto the teeth using a microbrush, a spray device, and/or a dental applicator tip. All areas of the teeth, including pits and fissures, may be covered with the dental coating composition. In some embodiments, the dental coating composition is applied evenly over the teeth surfaces.

At step 208, the dental coating composition can be cured. Various curing methods may be used (e.g., chemical curing, light curing, air dry, etc.). For example, the dental coating composition may be allowed to set on the teeth for a predetermined period of time at room temperature, such as by ensuring that a patient does not eat, drink, or otherwise disrupt the dental coating composition as the dental coating composition dries. In some embodiments, the dental coating composition may be cured using light. For example, the dental coating composition may include one or more photoinitiators. The teeth may be exposed to a curing light (e.g., an LED light, a halogen light, a UV light, etc.) for a predetermined amount of time. For example, the teeth may be exposed to an LED light with a wavelength of about 450-480 nm for about 20-40 seconds per tooth surface. Photoinitiators in the dental coating composition may absorb radiation from the curing light and responsively form reactive species. The reactive species may then initiate reactions (e.g., polymerization chain reactions) that cause the dental coating composition to harden and adhere to the teeth.

After the dental coating composition has been cured, the patient may be advised to refrain from eating and/or drinking for a predetermined amount of time (e.g., at least thirty minutes) to ensure optimal adhesion of the dental coating composition to the teeth. The patient may also be advised to avoid abrasive toothpaste or acidic foods within 24 hours of application. The coating may be configured to provide protection against tooth decay for at least six months up to several years (e.g., two years, three years), depending on individual oral conditions. The integrity of the coating may be assessed during regular dental check-up visits and may be reapplied as needed using the method described above.

In some embodiments, a dental coating kit can include a dental coating composition disclosed herein, an application device for applying the dental coating composition to teeth, and/or instructions for applying the dental coating composition to teeth (e.g., how to apply to teeth, how to cure, etc.).

### Example

20.0 g of chitosan was dissolved in 30.0 g of water and 10.0 g of ethanol with moderate stirring (about 300-500 rpm). 15.0 g of nanohydroxyapatite was dispersed in water and ethanol and subsequently added to the chitosan solution. While continuing to stir the solution, 2.0 g of PLGA-encapsulated sodium fluoride and 0.2 g of nisin were added. 5 g of phosphate buffer, 0.5 g of carbomer, and 0.2 g of potassium sorbate were also added to the solution while continuing to stir. 0.2 g of peppermint oil, 0.2 g of a camphorquinone-based photoinitiator, and 16.7 g of glycerol were also stirred into the solution.

To fully homogenize the solution to form the dental coating composition, the solution was mixed at a low speed (about 300-500 rpm) for about 10-15 minutes. The resulting dental coating composition was visually inspected to confirm the absence of lumps or phase separation. The dental coating composition appeared off-white and slightly opaque. The dental coating composition was mint-scented. The pH of the dental coating composition was 7.0, and the viscosity of the dental coating composition at 25° C. was about 2,500-3,000 cP as measured using a Brookfield LV-4 spindle at 20 rpm.

The final composition of the dental coating composition is shown in the following table:

TABLE 1

| | | Wt. % of |
|---|---|---|
| Component | Amount (g) | Composition |
| Distilled water | 30.0 | 30.0% |
| Ethanol | 10.0 | 10.0% |
| Chitosan (~80% deacetylation) | 20.0 | 20.0% |
| Nanohydroxyapatite (D50 ~40-80 nm) | 15.0 | 15.0% |
| Encapsulated sodium fluoride in PLGA | 2.0 | 2.0% |
| Nisin | 0.2 | 0.2% |
| Phosphate buffer (7.0) | 5.0 | 5.0% |
| Carbomer | 0.5 | 0.5% |
| Potassium sorbate | 0.2 | 0.2% |
| Peppermint oil | 0.2 | 0.2% |
| Camphorquinone & amine co-initiator | 0.2 | 0.2% |
| Glycerol | 16.7 | 16.7% |
| TOTAL | 100.0 | 100.0% |

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “powder(s)” and “particle(s)” used herein are equivalent, except that a single powder refers to a plurality of particles. This disclosure can apply to a wide range of particles and powders.

Reference to “about” a value or parameter herein includes (and describes) variations that are directed to that value or parameter per se. For example, description referring to “about X” includes description of “X”. In addition, reference to phrases “less than”, “greater than”, “at most”, “at least”, “less than or equal to”, “greater than or equal to”, or other similar phrases followed by a string of values or parameters is meant to apply the phrase to each value or parameter in the string of values or parameters. For example, a statement that a formulation has at most about 10 wt. %, about 15 wt. %, or about 20 wt. % of a component is meant to mean that the formulation has at most about 10 wt. %, at most about 15 wt. %, or at most about 20 wt. % of a component.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text. The numerical ranges disclosed inherently support any range or value within the disclosed numerical ranges, including the endpoints, even though a precise range limitation is not stated verbatim in the specification because this disclosure can be practiced throughout the disclosed numerical ranges.

The above description is presented to enable a person skilled in the art to make and use the disclosure and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown but is to be accorded the widest scope consistent with the principles and features disclosed herein.

1. A dental coating composition comprising:
nanohydroxyapatite;
at least one fluoride-releasing agent;
at least one antimicrobial peptide;
at least one bioadhesive polymer; and
at least one pH buffering agent.

2. The dental coating composition of claim 1, comprising 1-30 wt. % the nanohydroxyapatite.

3. The dental coating composition of claim 1, wherein the nanohydroxyapatite has a D50 particle diameter of about 20-100 nm as measured by dynamic light scattering at 25° C. in an aqueous dispersion.

4. The dental coating composition of claim 1, comprising 0.1-5 wt. % the at least one fluoride-releasing agent.

5. The dental coating composition of claim 1, wherein the at least one fluoride-releasing agent comprises sodium fluoride, stannous fluoride, sodium monofluorophosphate, potassium fluoride, amine fluorides, or combinations thereof.

6. The dental coating composition of claim 1, wherein the at least one fluoride-releasing agent comprises at least one fluoride compound encapsulated within a biodegradable polymer.

7. The dental coating composition of claim 6, wherein the biodegradable polymer comprises polylactic-co-glycolic acid.

8. The dental coating composition of claim 1, comprising 0.01-1 wt. % the at least one antimicrobial peptide.

9. The dental coating composition of claim 1, wherein the at least one antimicrobial peptide comprises nisin, defensins, or combinations thereof.

10. The dental coating composition of claim 1, wherein the at least one antimicrobial peptide targets at least one of Streptococcus mutans, Lactobacillus acidophilus, and Actinomyces viscosus.

11. The dental coating composition of claim 1, comprising 10-50 wt. % the at least one bioadhesive polymer.

12. The dental coating composition of claim 1, wherein the at least one bioadhesive polymer comprises chitosan, polyvinyl alcohol, polyacrylic acid, a methacrylate derivative, or combinations thereof.

13. The dental coating composition of claim 1, comprising 1-10 wt. % the at least one pH buffering agent.

14. The dental coating composition of claim 1, wherein the at least one pH buffering agent comprises calcium carbonate, sodium bicarbonate, a phosphate buffer, or combinations thereof.

15. The dental coating composition of claim 1, wherein the pH buffering agent is configured to maintain an oral pH of 6.5-7.5.

16. The dental coating composition of claim 1, comprising at least one flavoring agent.

17. The dental coating composition of claim 16, wherein the at least one flavoring agent comprises peppermint oil, spearmint oil, cinnamon oil, vanilla extract, citrus extracts, menthol, eucalyptol, or combinations thereof.

18. The dental coating composition of claim 1, comprising at least one coloring agent.

19. The dental coating composition of claim 18, wherein the at least one coloring agent comprises titanium dioxide, iron oxides, or combinations thereof.

20. The dental coating composition of claim 1, comprising at least one preservative.

21. The dental coating composition of claim 20, wherein the at least one preservative comprises potassium sorbate, phenoxyethanol, parabens, or combinations thereof.

22. The dental coating composition of claim 1, comprising at least one photoinitiator.

23. The dental coating composition of claim 22, wherein the at least one photoinitiator comprises camphorquinone, phenylpropanedione (PPD), trimethylbenzoyl diphenylphosphine oxide (TPO), phenylbis(2,4,6-trimethylbenzoyl)-phosphine oxide (BAPO), or combinations thereof.

24. The dental coating composition of claim 1, comprising at least one thickening agent.

25. The dental coating composition of claim 24, wherein the at least one thickening agent comprises carbomers, cellulose derivatives, fumed silica, or combinations thereof.

26. The dental coating composition of claim 1, comprising at least one plasticizer.

27. The dental coating composition of claim 26, wherein the at least one plasticizer comprises glycerol.

28. The dental coating composition of claim 1, comprising at least one solvent.

29. The dental coating composition of claim 28, comprising 10-60% the at least one solvent.

30. The dental coating composition of claim 28, wherein the at least one solvent comprises distilled water, ethanol, dichloromethane, ethyl acetate, or combinations thereof.

31. The dental coating composition of claim 1, wherein a viscosity of the dental coating composition is about 1,000-5,000 cP.

32. The dental coating composition of claim 1, wherein the dental coating composition is configured to provide protection against tooth decay for at least six months.

33. The dental coating composition of claim 32, wherein the dental coating composition is configured to provide protection against tooth decay for at least twelve months.

34. The dental coating composition of claim 1, wherein an adhesion strength of the dental coating composition to teeth is about 5-15 MPa.

35. The dental coating composition of claim 1, wherein the dental coating composition is configured to release about 0.1-1 ppm fluoride per day into a mouth of a patient.

36. The dental coating composition of claim 1, wherein patients treated with the dental coating composition experience about a 20-60% reduction in dental caries over a 12-month period.

37. A method of applying a dental coating composition to teeth of a patient, the method comprising:
coating the teeth with the dental coating composition of claim 1; and
curing the dental coating composition.

38. The method of claim 37, wherein coating the teeth with the dental coating composition comprises applying the dental coating composition to the teeth with a microbrush, an applicator tip, or a spray device.

39. The method of claim 37, wherein curing the dental coating composition comprises allowing the dental coating composition to set for a predetermined amount of time at room temperature.

40. The method of claim 37, wherein curing the dental coating composition comprises exposing the dental coating composition to a curing light.

41. The method of claim 40, wherein the curing light comprises an LED light, a halogen light, a UV light, or combinations thereof.

42. The method of claim 37, comprising:
prior to coating the teeth with the dental coating composition, removing plaque and/or tartar from the teeth.

43. The method of claim 37, comprising:
prior to coating the teeth with the dental coating composition, applying an etching solution to the teeth.

44. The method of claim 43, wherein the etching solution comprises 10% phosphoric acid.

45. The method of claim 43, wherein the etching solution is applied for 15-30 seconds.

46. A method of preparing the dental coating composition of claim 1, the method comprising:
dissolving the at least one bioadhesive polymer in a solvent to form a solution;
adding the nanohydroxyapatite, the at least one fluoride-releasing agent, the antimicrobial peptides, and the at least one pH buffering agent to the solution; and
mixing the solution to form the dental coating composition.

47. The method of claim 46, wherein the solvent comprises distilled water, ethanol, or combinations thereof.

48. The method of claim 46, comprising:
prior to adding the at least one fluoride-releasing agent to the solution, encapsulating at least one fluoride compound in a biodegradable polymer to form the at least one fluoride-releasing agent.

49. The method of claim 46, comprising:
adding at least one thickening agent to the solution.

50. The method of claim 46, comprising:
sterilizing the dental coating composition.

51. The method of claim 50, wherein sterilizing the dental coating composition comprises irradiating the dental coating composition with gamma radiation, autoclaving the dental coating composition, or performing aseptic processing.
